(19) United States
(12) Patent Application Publication
(10) Pub. No.: US 2005/0096264 A1
(43) Pub. Date: May 5, 2005

(54) Methods for treating obesity and related conditions with glycoprotein hormone beta family hormones

(76) Inventors: Lynn MacDonald, White Plains, NY (US); Mark W. Sleeman, Mahopac, NY (US); Andrew J. Murphy, Croton-on-Hudson, NY (US)

Correspondence Address: REGENERON PHARMACEUTICALS, INC, 777 OLD SAW MILL RIVER ROAD, TARRYTOWN, NY, 10591, US

(21) Appl. No.: 10/990,058

(22) Filed: Nov. 16, 2004

Related U.S. Application Data
(63) Continuation-in-part of application No. 10/373,617, filed on Feb. 25, 2003.

(51) Int. Cl. A61K 38/22

(52) U.S. Cl. 514/8

(57) ABSTRACT

A method of treating or ameliorating an orphan glycoprotein hormone (OGH)-related condition or a condition which responds to OGH administration, comprising administering an OGH-related compound to a subject in need thereof.

### CROSS-REFERENCE TO RELATED APPLICATIONS

The application is a continuation-in-part of U.S. Ser. No. 10/373,617 filed 25 Feb. 2003, which claims the benefit under 35 USC § 119(e) of U.S. Ser. No. 09/684,197 filed 6 Oct. 2000, which applications are herein specifically incorporated by reference in their entirety.

### BACKGROUND

1. Field of the Invention

This invention relates to therapeutic methods for treating orphan glycoprotein hormone (OGH, also called GPB5) related conditions, as well as conditions involving related glycoprotein family members.

2. Description of Related Art

Thyroid stimulating hormone (TSH) and the TSH receptor (TSHR) are key proteins in the control of thyroid function. TSH (thyrotropin) synthesis and release is stimulated by hypothalamic thyrotropin releasing hormone (TRH) and is downregulated (inhibited) by thyroid hormone in a classic endocrine negative feedback loop. The specificity inherent in TSH resides in its unique b-subunit that heterodimerizes with a common a-subunit which it shares with the other glycoprotein hormones (i.e., follicle stimulating hormone (FSH), luteinizing hormone (LH) and chorionic gonadotropin (CG)). The primary physiological actions of TSH on the thyroid are stimulation of the synthesis and release of 3,5,3′,5′-tetraiodo-L-thyronine (T4) and 3, 5, 3′-triiodo-L-thyronine (T3), (together termed thyroid hormone) and promotion of thyroid growth; for instance it has been shown that thyroid development is arrested in mice with targeted disruption of the common a subunit (Kendall et al. (1995) Genes Dev 9:2007-2019).

### BRIEF SUMMARY

The present invention provides therapeutic methods for treating a variety of obesity-related conditions, including for decreasing body weight, body fat, serum cholesterol, serum triglycerides, and blood glucose in a subject. The present invention provides methods of treating obesity and obesity-related conditions mediated through the thyroid axis without accompanying cardiovascular toxicity seen in prior art therapeutics, for example, with the administration of T3, T4, or non-selective thyroid hormone agonists.

In a first aspect, the invention features a method of treating obesity, comprising administering an OGH-related compound to a subject in need thereof. In one embodiment, the OGH-related compound is a compound capable of activating thyroid stimulating hormone (TSH) receptor, such as the glycoprotein beta subunit OGH, or an OGH variant or fragment thereof. An OGH-related compound may include the appropriate alpha subunit, e.g. OGH and α2, and active fragments and variants thereof. Preferably, the subject being treated is a patient suffering from obesity or morbid obesity, or determined to be obese by conventional methods known to those of skill in the art. Obesity is defined by body mass index (BMI) which is derived from weight (kg)/Height (m²); normal BMI=18.5-25; Overweight=25-30; Obese=30-40 and morbidly obese 40+ (Source: World Obesity Congress 2004, Washington D.C.).

In a second aspect, the invention features a method of decreasing weight, comprising administering a therapeutically effective amount of an OGH-related compound to a subject in need thereof.

In a third aspect, the invention features a method of inducing resistance to weight gain related to ingestion of a high fat diet, the method comprising administering an amount of an OGH-related compound to a subject, wherein the subject gains less weight in response to a high fat diet relative to weight gain in the absence of an OGH-related molecule. A high fat diet being defined as one that consists of greater than 20 kcal % fat.

In a fourth aspect, the invention features a method of reducing blood glucose, comprising administering an amount of an OGH-related compound to a subject in need thereof.

In a fifth aspect, the invention features a method of reducing serum insulin, comprising administering an amount of an OGH-related compound to a subject in need thereof.

In a sixth aspect, the invention features a method of reducing serum cholesterol, comprising administering an amount of an OGH-related compound to a subject in need thereof.

In a seventh aspect, the invention features a method of reducing serum triglycerides, comprising administering an amount of an OGH-related compound to a subject in need thereof.

In an eighth aspect, the invention features a method of improving, reducing, or ameliorating obesity-related conditions, comprising administering an amount of an OGH-related compound to a subject in need thereof. The obesity-related conditions which are improved, reduced, or ameliorated include one or more of body weight, serum insulin level, serum cholesterol level, and/or serum triglyceride level.

In a tenth aspect, the invention features a pharmaceutical composition comprising an agent capable of activating the TSH receptor and a pharmaceutically acceptable carrier. In specific embodiments, the agent is the glycoprotein hormone beta subunit OGH, an OGH variant, or fragment thereof. In another embodiment, the agent is OGH and α2, and active fragments and variants thereof. In a preferred embodiment, the pharmaceutical composition of the invention is a sustained release composition.

Other objects and advantages will become apparent from a review of the ensuing detailed description.

### BRIEF DESCRIPTION OF THE FIGURES

FIG. 1. OGH-TG design. Top line: OGH-TG. Bottom line: wt Rosa26. Arrow labeled OGH: adenovirus splice acceptor, mouse OGH cDNA coding sequence and rabbit b-globin polyA. Arrow labeled PGK-neo: mouse PGK promoter, Tn5 neo and gene mouse PGK polyA. Restriction sites and probe used for Southern are shown.

FIG. 2A-F. Body weight, body fat, and VO2 of male wildtype or OGH transgenic mice. Mice weighed once a week from birth (FIG. 2A) and, starting at 9 weeks of age, for 12 weeks on a high-fat diet for (FIG. 2B). Changes in body weight on high-fat diet shown as % difference from the first day of the diet. Measurements of body fat (FIG. 2C-D) taken immediately before, and again after 6 and 12 weeks on high fat diet. Metabolic rate (FIG. 2E-F) before, and after 6 weeks on high fat diet. Data expressed as Mean±SEM. One-way ANOVA: *-difference from wild type littermate controls, P<0.05.

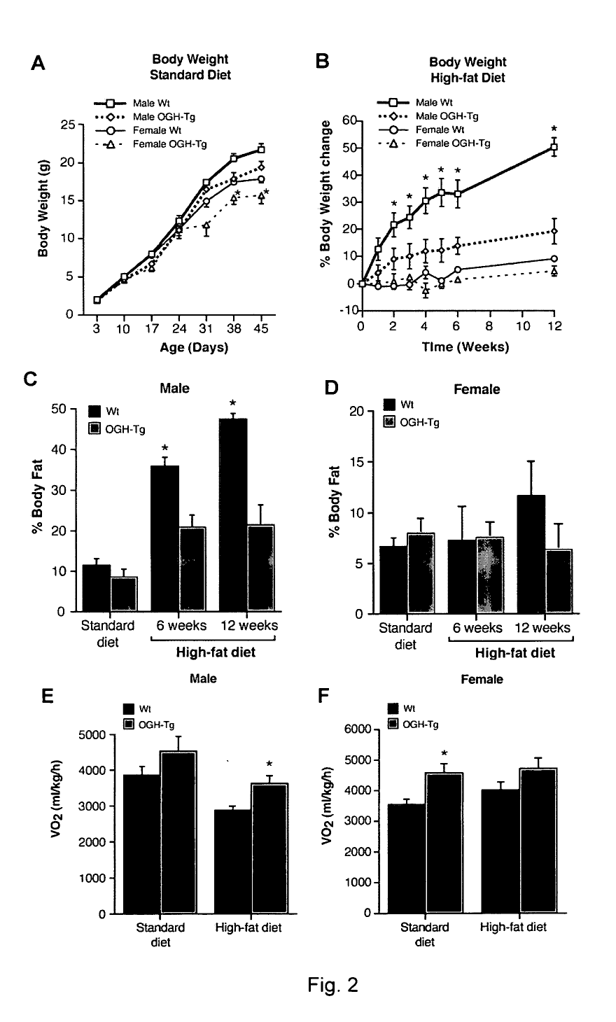

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference in their entirety.

Definitions

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the term “OGH-mediated condition” is meant a condition which involves the OGH protein. For example, a condition which can be improved, ameliorated, or reduced by administration an OGH-related protein includes, for example, weight loss, obesity, metabolic rate, blood glucose, cholesterol, and triglyceride levels.

By the term “OGH-related compound” is meant a glycoprotein hormone protein or variant, or fragment thereof, which is capable of activating the thyroid stimulating hormone (TSH) receptor when present in a heterodimer with its alpha subunit. Generally, an OGH-related compound will mean the glycoprotein beta subunit is OGH, or an OGH variant or fragment thereof. An OGH-related compound may also include the appropriate alpha subunit, e.g. OGH and α2, and active fragments and variants thereof.

General Description

Clinically, thyroid hormone itself is used primarily as a replacement therapy for the patients with hypothyroidism, and it has been considered as a possible therapeutic for weight reduction (due to its ability to increase metabolism and energy expenditures), for lowering cholesterol, and even to build bone (in osteoporosis). The major hurdle in this approach has been the cardiovascular toxicity observed following administration of the endogenous ligands T4 and T3 or non-selective thyroid hormone agonists. The two major subtypes of the thyroid hormone receptors (TR) that mediate these responses are the α (TRα) and β (TRβ) which are the products of different genes and are also differentially processed to each yield 2 isoforms. It has been argued that modulation of heart rate and rhythm is mediated predominantly through activation of the TRα₁ (see, for example, Ribiero et al. (2001) J Clin Invest 108:97-105), and as a result recent pharmaceutical research efforts have tended to focus on developing specific TRβ₁ agonists (see, for example, Grover et al. (2003) Proc Natl Acad Sci USA 100:10067-72; Ye et al. (2003) J Med Chem 46:1580-8).

A human gene encoding a homologue of the glycoprotein hormone beta subunits was identified and named orphan glycoprotein hormone (OGH). OGH knockout/lacZ knock-in mice (Ogh^{-/-}) as well as mice that globally over express OGH (OGH-TG) were generated to study the function of OGH. Subsequent studies by Hsu et al. (2002) Mol Endocrinol 16:1538-8, termed the protein GPB5, and described a new human homologue of the common glycoprotein hormone alpha subunit, called GPA2. It was shown that GPB5 and GPA2 heterodimerize and that the heterodimer activates the TSHR, and the term thyrostimulin was coined to describe the GPB5/GPA2 heterodimer (Nakabayashi et al. (2002) J Clin Invest 109:1445-52.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In one embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In one embodiment of the invention, an OGH-related compound is formulated in a sustained-release formulation. As shown in the experiments described below, the desirable effects achieved with a low-level activation of the thyroid axis are accompanied without the cardiotoxic effects seen with adminstration of T3 and T4. Accordingly, these results support a sustained-release formulation for long-term administration of low levels of the OGH-related compounds described above. Sustained release formulations for derlivery of biologically active peptides are known to the art. For example, U.S. Pat. No. 6,740,634, herein specifically incorporated by reference in its entirety, describes a sustained-release formulation containing a hydroxynaphtoic acid salt of a biologically active substance and a biodegradable polymer. U.S. Pat. No. 6,699,500, herein specifically incorporated by reference in its entirety, discloses a sustained-release formulation capable of releasing a physiologically active substance over a period of at least 5 months.

The amount of the active agent of the invention which will be effective in the treatment of a OGH-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Cellular Transfection and Gene Therapy

The present invention encompasses the use of nucleic acids encoding the OGH-related compounds of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.”

In another aspect, the invention provides a method of treating OGH-mediated conditions, such as obesity, in a subject comprising transfecting a cell with a nucleic acid encoding protein of the invention, wherein the nucleic acid comprises an inducible promoter operably linked to the nucleic acid encoding the protein. For gene therapy procedures in the treatment or prevention of human disease, see for example, Van Brunt (1998) Biotechnology 6:1149-1154.

Combination Therapies

In numerous embodiments, the fusion proteins of the present invention may be administered in combination with one or more additional compounds or therapies. Combination therapy includes administration of a single pharmaceutical dosage formulation which contains a compound capable of activating the TSH receptor and one or more additional hypoglycemic agent or weight loss agent; as well as administration of a fusion protein and one or more additional hypoglycemic agent or weight loss agent in its own separate pharmaceutical dosage formulation. For example, an OGH-related compound of the invention and a hypoglycemic agent can be administered to the patient together in a single oral dosage composition such as a tablet or capsule, or each agent administered in separate oral dosage formulations. Where separate dosage formulations are used, the OGH-related compound and one or more additional hypoglycemic agents can be administered at essentially the same time, i.e., concurrently, or at separately staggered times, i.e., sequentially.

An examples of such weight loss agents is Axokine® (Regeneron) Examples of such hypoglycemic agents include: insulin; biguanidines, such as metformin Glucophage® (BMS), and buformin; sulfonylureas, such as acetohexamide, Diabinese® (Pfizer), Amaryl® (Aventis), Glynase Pres Tabs® (Pharmacia), Glucotrol XL® (Roering Pfizer), tolazamide, tolbutamide, DiaBeta® (Hoechst), Glucotrol® (Pfizer) and glyclazide; thiazolidinediones, such as Rezulin® (Park Davis), Actos® (Tekada), and Avandia® (GSK); α-glycosidase inhibitors, such as Precose® (Bayer) and Glyset® (Bayer); Meglitinide such as Prandin® (Novo Nordisk); Glucose Elevating Agents such as Glucagon® (Lilly); and β₃ adrenoreceptor agonists such as CL-316,243.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a transgenic knock-out animal having a modified endogenous OGH gene. A transgenic animal can be produced by introducing nucleic acid into the male pronuclei of a fertilized oocyte, e.g., by microinjection, retroviral infection, and allowing the oocyte to develop in a pseudopregnant female foster animal. Still further, the invention contemplates a transgenic animal having an exogenous OGH gene generated by introduction of any OGH-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the OGH protein to particular cells.

It is useful to provide non-human transgenic animals to assay in vivo OGH protein function, including receptor interaction, the effect of specific mutant OGH proteins on OGH protein function and binding partner interaction, and the effect of chimeric OGH proteins. It is also possible to assess the effect of null mutations,that is mutations that substantially or completely eliminate one or more OGH protein functions.

Specific Embodiments

The experiments described below show that OGH-TG mice are resistant to diet-induced obesity, even though they ate more compared to wild-type mice. Their failure to gain weight correlates with an increased metabolic rate as evidenced by their increased consumption of oxygen and production of carbon dioxide. The increased metabolic rate of OGH-TG mice is due to the ability of OGH (GPB5) to heterodimerize with GPA2 and comprise thyrostimulin which can then activate the TSH receptor, resulting in about 1.5 to 2.5 fold elevations in circulating T3 and T4 in the OGH-TG mice as compared to wild-type mice.

OGH-TG mice also have significantly lower levels of circulating cholesterol. However, unlike mice treated with exogenous thyroid hormone (Weiss et al. (2002) Am J Physiol Endocrinol Metab 283:E428035), OGH-TG mice do not exhibit significantly elevated heart rates. One possible explanation of this difference may reside in the different levels of thyroid hormone in the two scenarios. For instance, in one study (Weiss et al. (2002) supra) in which thyroid hormone treatment of wild type mice resulted in a 43% increase in heart rate, the level of circulating T4 was increased over 10 fold. Likewise, in another study (Johansson et al. (1997) Acta Physiol Scand 160:133-8) a fivefold increase in circulating T3 levels produced a 14-20% increase in heart rate after 3 or 4 days. In contrast, T4 levels in OGH-TG mice are maintained at a level of only approximately twofold higher than in wild-type mice, and T3 levels are only about 50% higher (Table 1). The experiments suggest that constitutive low-level activation of the thyroid axis (via OGH or other means) may provide a beneficial therapeutic approach for combating diet-induced obesity.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Subunit Structure of Human OGH

Epitope tagged versions of the indicated proteins human OGH (hOGH), hCG (human Chorionic Gonadotropin β subunit; Genebank acc#NP_{—}000728) and alpha (α) (the human, common alpha subunit of the glycoprotein hormones; Genebank acc#NP_{—}000726) were expressed in COS cells using standard procedures known to the skilled artisan.

Media supernatants obtained from the COS transfectants described supra were either run directly (15 μl per lane, Panel A and Panel B) on reducing, denaturing acrylamide gels (4-20% gradient, Novex) or run after immunoprecipitation (Panel C) as follows: One ml of culture supernatant from each transfection was chilled on ice and mixed with 0.5 ml of cold TBS, 2.2 μg of M2 anti-FLAG monoclonal antibody (Sigma) and 0.05 ml of protein G sepharose beads (Pharmacia). The mixture was gently mixed at 4° C. for 2.5 hours. The beads were collected by centrifugation, washed 3 times with TBS plus 1% NP40 and proteins were eluted from pelleted beads with 30 μl of loading buffer. 15 μl of the recovered proteins were loaded per gel lane.

Gels were transferred overnight by standard procedures, blocked with 10% non-fat dried milk and probed with either anti-HA (0.5 μg/ml monoclonal 12CA5, Boehringer-Mannheim) or anti-FLAG (0.44 μg/ml monoclonal M2, Sigma) for 1 hour, washed, probed with a secondary antibody (0.077 μg/ml HRP conjugated anti-mouse IgG, Promega), washed and developed with the ECL luminescence kit as per manufacturers instructions (NEN).

Flag-tagged hOGH immunoprecipitated both co-expressed HA-tagged common glycoprotein hormone alpha (a) subunit and HA-tagged hOGH with efficiencies roughly comparable to that at which FLAG-tagged hCG immunoprecipitated common glycoprotein hormone a subunit. These results indicate that hOGH can form homo-dimers (or higher order homomeric structures) as well as hetero-dimers (or higher order heteromeric structures) with the common glycoprotein hormone a subunit. The homo-multimeric and hetero-multimeric forms are likely to have different biological activities perhaps binding to different receptors or acting as an agonist/antagonist pair on the same receptor or group of receptors.

### Example 2

Generation of OGH Trangenic Mice

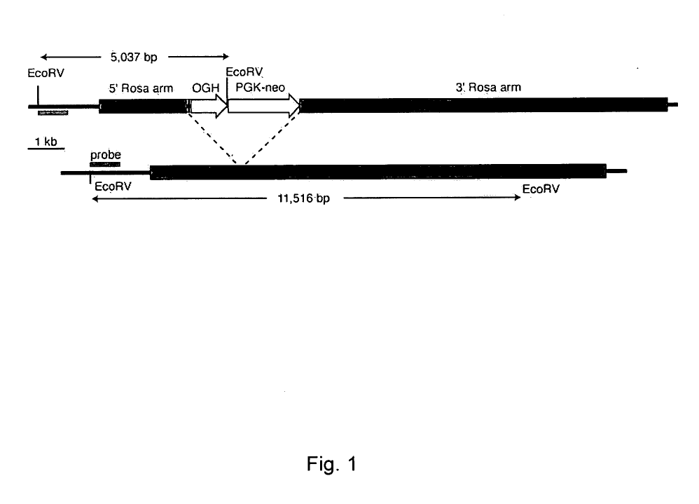

To globally over-express OGH, the mouse OGH cDNA coding sequence was “knocked-in” to the Rosa26 locus (Friedrich et al. (1991) Genes Dev 5:1513-23). To generate OGH knockout mice in which the the coding region of the OGH gene was precisely deleted (from initiation to termination codon) in ES cells and replaced with a lacZ reporter gene and neomycin selectable marker (FIG. 1) using the VelociGene® technology (Valenzuela et al. (2003) Nat Biotechnol 21:652-9). Correctly targeted ES cells and mice were identified by a real-time PCR-based “loss-of-native-allele” assay. Heterozygous mice were backcrossed to C57BL/6-J to generate N2 breeding heterozygote pairs that were used to generate homozygous null N2F2 mice. Correct targeting was reconfirmed in these mice by Southern blot analysis. All experiments reported were conducted on such N2F2 littermates that were housed in 12 hours of light per day (0700 h-1900 h) in a temperature-controlled environment. All procedures were conducted in compliance with protocols approved by the Regeneron Institutional Animal Care and Use Committee. Animals had free access to either standard chow (Purina # 5020; St Louis, Mo.) or high fat diet (45% fat, Harlan Teklad # 93075; Madison, Wis.) as specified.

Deletion and expression pattern of endogenous OGH gene. A normal birth ratio of wild-type (Ogh^{+/+}), heterozygous (Ogh^{+/-}) and homozygous (Ogh^{-/-}) mice was observed, and the male and female Ogh^{-/-} mice appeared grossly normal and reached normal development milestones during the first 8 weeks of age. To assess expression of the endogenous OGH gene, whole-body lacZ analysis was performed on adult heterozygous mice. No site of abundant expression was found, but a few sites of low-level expression were noted. LacZ was detected over background in a specific region of the brain around the caudal fasciculus retroflexus, in retina and in the seminiferous tubules of the testes. In contrast to adult animals, newborns showed strong lacZ staining in what appear to be a subset of salivary glands as well as apparent ducts in the palate. The Ogh^{-/-} mice were of normal body weight, composition and metabolic parameters.

Global Overexpression OGH. A high level of mRNA expression from the Rosa-OGH transgene in F1 heterozygous mice was found by real-time quantitative RT-PCR (TaqMan™) analysis of three arbitrarily chosen tissues, heart, kidney and liver. In contrast, the levels of naturally expressed OGH mRNA were found to be very low (less than 1 mRNA molecule for every five cells) in these three tissues as well as about thirty other mouse tissues and human tissues examined, consistent with the rare, limited expression of the lacZ reporter gene in Ogh^{-/-} mice.

While a normal birth ratio of wild-type and heterozygous (OGH-TG) mice was observed, all OGH-TG mice had a shortened snout. This phenotype is first apparent at about 10 days of age and becomes more prominent as the animals mature. We measured the lengths and widths of facial bones of adult OGH-TG and wild type animals and determined that a significant shortening of the nasal and frontal bones occurs in the transgenics whereas no difference was seen in the length or width of the parietal bone. No other skeletal defect was detected in OGH-TG mice.

LacZ and histological analyses. Whole mount lacZ analysis was conducted as previously described (Suri et al. (1996) supra). For histomorphometric analysis, skulls were cleared of surrounding tissue and stained with alcian blue and alizarin red. Digital photographs were taken of the dorsal aspect of the skull and measurements were made using the public domain image analysis program, NIH Image. Measurements of the lengths of the nasal, frontal and parietal bones were performed along the sagittal suture. The width of the parietal bone was measured along a line perpendicular to the mid-sagittal plane and extending from the sagittal suture to the lateral crest of the parietal bone.

### Example 3

Serum Measurements.

Basal serum samples were taken between 10 am and 12 noon, after an overnight fast, and analyzed for glucose, triglycerides, cholesterol, and T3 utilizing the Bayer 1650 blood chemistry analyzer (Bayer, Tarrytown N.Y.). Insulin levels were analyzed by LincoPlex (Linco, St. Charles, Mo.). TSH and T4 levels were analyzed by radioimmunoassay (Dr. A. F. Parlow, UCLA-REI, CA).

We determined levels of glucose, lipids and several relevant hormones in the blood of 5-month-old OGH-TG mice on a standard diet (Table 1). Male and female OGH-TG mice weighed significantly less than their respective wild-type littermates. Blood glucose, triglycerides and insulin levels were reduced in male OGH-TG mice consistent with reduced body weight, and total serum cholesterol was significantly reduced in both male and female OGH-TG animals. Similar changes in serum chemistry were seen in OGH-TG mice compared to their wild-type littermates when both were placed on a high fat diet (data not shown).

Importantly, basal T3 and T4 levels were significantly increased about 1.5-2.5 fold in both male and female OGH-TG mice compared to their wild-type littermates, while TSH levels were reduced or unchanged (Table 1), suggesting mild negative feedback due to the increases in T3 and T4. These increased T3 and T4 are likely due to a direct action on the thyroid, as others have reported (Nakabayashi et al. (2002) supra), and we have independently confirmed that OGH/GPB5 is able to activate the TSHR, when co-expressed with a distant homologue of the common glycoprotein hormone alpha subunit, termed GPA2 or Zsig51. Thus, we infer that the global over-expression of OGH/GPB5 in OGH-TG animals includes a site, or sites, which also express GPA2. Many, if not all, of the phenotypic differences seen in the OGH-TG mice may be attributed to pleiotropic effects resulting from constitute low-level activation of the thyroid axis due to the OGH.

TABLE 1

| | Male | | Female | |
|---|---|---|---|---|
| | Wt | OGH-Tg | Wt | OGH-Tg |
| Body | 31.6 ± 3.1 | 26.8 ± 3.6* | 26.4 ± 3.2 | 20.5 ± 2.5* |
| weight (g) | | | | |
| Glucose | 277 ± 10 | 214 ± 25* | 214 ± 10 | 213 ± 17 |
| (mg/dL) | | | | |
| Triglycerides | 121 ± 15 | 76 ± 7* | 80 ± 11 | 76 ± 6 |
| (mg/dL) | | | | |
| Cholesterol | 118 ± 9 | 71 ± 7* | 90 ± 5 | 69 ± 6* |
| (mg/dL) | | | | |
| Insulin | 2.5 ± 0.18 | 1.9 ± 0.15* | No data | No data |
| (ng/mL) | | | | |
| TSH | 174 ± 13 | 132 ± 5* | 95 ± 3 | 93 ± 9 |
| (ng/mL) | | | | |
| T3 (ng/dL) | 84 ± 5 | 130 ± 10* | 69 ± 7 | 119 ± 14* |
| T4 (ng/dL) | 2.8 ± 0.2 | 6.9 ± 1.1* | 3.5 ± 0.3 | 5.7 ± 1.0* |

### Example 4

Indirect Calorimetry.

Metabolic parameters were obtained using an Oxymax (Columbus Instruments International Corp., Columbus, Ohio) open circuit indirect calorimetry system. The system was calibrated against a standard gas mixture to measure O₂ consumed (ml/kg/hr) and CO₂ generated (ml/kg/hr) by each animal at 57 minute intervals for a 72 hour period. Energy expenditure was calculated as the product of calorific value of oxygen (=3.815+1.232×respiratory quotient) and the volume of O₂ consumed and was normalized for body weight (kg). The first 2 hours of measurement was used as a period of adaptation for the animals and metabolic rate and activity were evaluated over the subsequent 70 hour period.

Metabolic analysis. We assessed metabolic parameters by indirect calorimetry of OGH-TG and age-matched wild type mice while on a standard diet and following six weeks on a high-fat diet. Both male and female OGH-TG mice consumed more oxygen per unit body weight than did wild type controls under both conditions (FIG. 1E-F). The amount of carbon dioxide produced was similarly increased in OGH-TG animals.

### Example 5

Body Composition.

Body composition was measured by Dual Energy X-ray Absorptiometry (DEXA) using the pDEXA Sabre Bone Densitometer (Norland Medical Systems; Fort Atkinson, Wis.). Mice were fasted for 4 hours and anesthetized with isoflurane before scanning. The entire body of each mouse was scanned twice. DEXA scans were performed 1 week before metabolic measurements to allow for recovery of food intake and body weight after anesthesia.

Analysis of body weight and composition. Mice homozygous for the Ogh deletion were identical to their wild-type littermates with respect to overall appearance, body weight, body composition (as measured by pDEXA), metabolic parameters and response to high fat diet. Unlike the knockouts, the OGH-TG mice exhibited a decreased body weight that first appeared at about one month of age and persisted into adulthood (FIG. 1A). Because there was no overall difference in body length in these animals, the body weight difference was investigated to determine whether it was the result of altered caloric intake or metabolism. When food consumption was measured, it was deterermined that instead of eating less than their heavier, wildtype littermates, the OGH-TG mice actually tended to eat slightly more (data not shown). When placed on a high fat diet, male OGH-TG mice gained significantly less weight (14% increase after 12 weeks) compared to their wild type littermates (50% increase after 12 weeks; FIG. 1B). This difference was smaller in the female animals, as female wild type mice characteristically showed only a small (<10%) increase in body weight on the high fat diet. The difference in body weight gain reflected a marked difference in adiposity (FIG. 1C-D). The percent of total body weight comprised of adipose in wildtype mice increased from 10% on a standard diet to over 45% on the high fat diet, whereas the OGH-TG mice showed a much smaller increase in adiposity (from about 10% to 20% body fat). The body composition differences were similar, but less pronounced, in females. After 12 weeks on the high fat diet, the wild type female mice had twice the relative amount of fat as their OGH-TG littermates (12% vs. 6%).

### Example 6

Cardiovascular Telemetry.

Blood pressure and heart rate measurements were recorded in unrestrained, conscious 12-week-old male mice via implanted transmitters (PA-C20, Data Sciences International, Minneapolis, Minn.). Under isoflurane anesthesia, the carotid artery was surgically exposed and the transmitter catheter threaded through the carotid to lie open to the blood flow of the aorta. The catheter was anchored to the carotid via suture, and the attached transmitter implanted in a subcutaneous pocket on the left flank. Following surgery, the mice were allowed to recover for a period of 10 to 14 days before measurements were recorded. Data was sampled in 10 sec intervals every 5 min for three days, and analyzed using Dataquest software (Data Sciences, International, Minneapolis, Minn.). Data collected from each animal were condensed to single mean values for light and dark cycle heart rate, diastolic and systolic blood pressure with standard errors reflecting variability between study animals. Animals with suspicious pulse pressure readings (below 20 mm Hg) were checked for correct catheter placement, blood clots, or tissue blockage and removed from subsequent analysis, where applicable, at the conclusion of the study.

Cardiovascular telemetry. Most other approaches that activate the thyroid axis for the purpose of promoting weight loss have been associated with undesirable increases in heart rate and blood pressure. Heart rate and blood pressure of OGH-TG animals and wild type littermates were determined via radio-telemetry in conscious unrestrained animals. Three days of continuous measurement were condensed into light cycle and dark cycle averages. Although heart rate, diastolic blood pressure, systolic blood pressure and pulse pressure tended to be slightly elevated in OGH-TG mice, these parameters were within normal limits and the differences failed to reach statistical significance (Table 2).

Statistical analysis. Data is expressed as mean±s.e.m. Comparison of means was carried out using a t-test or analysis of variance (ANOVA) where appropriate using the program STATVIEW (SAS, Cary, N.C.). When a significant F ratio was obtained (significance P<0.05), post hoc analysis was conducted between groups using a multiple comparison procedure with Bonferroni/Dunn correction of means (ANOVA) or Dunnett post hoc comparison. P-values less than 0.05 were considered statistically significant and marked with an asterisk.

Deposit of Biological Material

The following clones were deposited with the American Type Culture Collection (ATCC®), 10801 University Boulevard, Manassas, Va. 20110-2209, on Sep. 24, 1999:

| Clone | Patent Deposit Designation |
|---|---|
| 325d23 human DNA insert in BAC vector | PTA-787 |
| 534i21 human DNA insert in BAC vector | PTA-788 |
| 399n04 human DNA insert in BAC vector | PTA-789 |

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating or ameliorating an orphan glycoprotein hormone (OGH)-related condition or a condition which responds to OGH administration, comprising administering an OGH-related compound to a subject in need thereof.

2. The method of claim 1, wherein the OGH-related compound is a compound capable of activating thyroid stimulating hormone (TSH) receptor.

3. The method of claim 2, wherein the OGH-related compound is selected from the group consisting of OGH, or an OGH variant or fragment thereof.

4. The method of claim 3, wherein the OGH-related compound further comprises glycoprotein hormone alpha subunit.

5. The method of claim 1, wherein the condition being treated is obesity, high serum cholesterol, high blood glucose, and/or high serum triglycerides.

6. A method of reducing body weight in an obese subject, comprising administering an OGH-related compound to the subject.

7. A method of inducing resistance to weight gain related to ingestion of a high fat diet, the method comprising administering an amount of an OGH-related compound to a subject, wherein the subject gains less weight in response to a high fat diet relative to weight gain in the absence of an OGH-related molecule.

8. The method of claim 7, wherein the high fat diet derives 20% or more of total calories from fat.

9. A method of reducing blood glucose, serum insulin, serum cholesterol, and/or serum triglyceride levels, comprising administering an amount of an OGH-related compound to a subject in need thereof.

10. A method of improving, reducing, or ameliorating obesity-related conditions, comprising administering an amount of an OGH-related compound to a subject in need thereof.

11. The method of claim 9, wherein the obesity-related conditions which are improved, reduced, or ameliorated include one or more of body weight, serum insulin level, serum cholesterol level, and/or serum triglyceride level.
